(19) United States
(12) Patent Application Publication
(10) Pub. No.: US 2024/0395365 A1
(43) Pub. Date: Nov. 28, 2024

(54) COMPUTER-READABLE RECORDING MEDIUM STORING SELF-SUPERVISED TRAINING PROGRAM, METHOD, AND DEVICE

(71) Applicant: Fujitsu Limited, Kawasaki-shi (JP)

(72) Inventor: Yasufumi SAKAI, Fuchu (JP)

(73) Assignee: Fujitsu Limited, Kawasaki-shi (JP)

(21) Appl. No.: 18/656,754

(22) Filed: May 7, 2024

(30) Foreign Application Priority Data
May 24, 2023 (JP) 2023-085717

(51) Int. Cl. G16C 20/30; G16C 20/70

(52) U.S. Cl. CPC G16C 20/30; G16C 20/70

(57) ABSTRACT

A non-transitory computer-readable recording medium stores a self-supervised training program for causing a computer to execute a process including: generating data that indicates a second molecule obtained by replacing each of a predetermined percentage of atoms among the atoms contained in a first molecule, with any of the atoms included in a group of a plurality of predefined types of the atoms; acquiring a prediction result by inputting the data that indicates the second molecule to a machine learning model that performs prediction regarding a molecular structure; and updating a parameter of the machine learning model, based on a comparison result between correct answer data that corresponds to the first molecule and the prediction result.

### CROSS-REFERENCE TO RELATED APPLICATION

This application is based upon and claims the benefit of priority of the prior Japanese Patent Application No. 2023-85717, filed on May 24, 2023, the entire contents of which are incorporated herein by reference.

### FIELD

The embodiments discussed herein are related to a self-supervised training program, a self-supervised training method, and a self-supervised training device.

### BACKGROUND

A neural network that predicts energy of a molecule, based on structural data of that molecule has been previously proposed. This neural network is trained by supervised training, using labeled data made up of “structure” and “energy”. This “energy” is calculated by, for example, a numerical calculation technique called density functional theory (DFT). The DFT is supposed to have a very long calculation time for molecular energy, and it sometimes takes half a day to three days to calculate the energy of one structure. For this reason, it is difficult to collect a large amount of labeled data for supervised training.

Japanese National Publication of International Patent Application No. 2021-518024 is disclosed as related art.

Kristof T. Schutt, Oliver T. Unke, Michael Gastegger, “Equivariant Message Passing for the Prediction of Tensorial Properties and Molecular Spectra”, PMLR, 2021, Johannes Gasteiger, Muhammed Shuaibi, Anuroop Sriram, Stephan Gunnemann, Zachary Ulissi, C. Lawrence Zitnick, Abhishek Das, “GemNet-OC: Developing Graph Neural Networks for Large and Diverse Molecular Simulation Datasets”, Transactions on Machine Learning Research, 2022, and Zaixi Zhang, Qi Liu, Shengyu Zhang, Chang-Yu Hsieh, Liang Shi, Chee-Kong Lee, “Graph Self-Supervised Learning for Optoelectronic Properties of Organic Semiconductors”, ICML, 2022 are also disclosed as related art.

However, in the previous technologies, there is a disadvantage that the neural network will be trained on information on “masking” that does not exist as an actual atom, which sometimes causes useless training to be performed and deteriorates the prediction accuracy of the neural network.

As one aspect, an object of the disclosed technology is to enhance the prediction accuracy of a neural network trained by self-supervised training.

### SUMMARY

According to an aspect of the embodiments, a non-transitory computer-readable recording medium stores a self-supervised training program for causing a computer to execute a process including: generating data that indicates a second molecule obtained by replacing each of a predetermined percentage of atoms among the atoms contained in a first molecule, with any of the atoms included in a group of a plurality of predefined types of the atoms; acquiring a prediction result by inputting the data that indicates the second molecule to a machine learning model that performs prediction regarding a molecular structure; and updating a parameter of the machine learning model, based on a comparison result between correct answer data that corresponds to the first molecule and the prediction result.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a diagram for explaining a neural network (NN) for molecular energy prediction;

FIG. 2 is a diagram for explaining transfer training;

FIG. 3 is a diagram for explaining self-supervised training designed for the NN for molecular energy prediction;

FIG. 4 is a diagram for explaining a problem of self-supervised training designed for the NN for molecular energy prediction;

FIG. 5 is a functional block diagram of a self-supervised training device;

FIG. 6 is a diagram for explaining processing of a self-supervised training device according to a first embodiment;

FIG. 7 is a block diagram illustrating a schematic configuration of a computer that functions as the self-supervised training device;

FIG. 8 is a flowchart illustrating an example of self-supervised training processing;

FIG. 9 is a diagram for explaining processing of a self-supervised training device according to a second embodiment;

FIG. 10 is a diagram for explaining an example of a configuration of an NN;

FIG. 11 is a diagram illustrating a validation result about an effect of the present technique;

FIG. 12 is a diagram illustrating a validation result about an effect of the present technique; and

FIG. 13 is a diagram illustrating a validation result about an effect of the present technique.

### DESCRIPTION OF EMBODIMENTS

Thus, there is a technique called self-supervised training that generates a correct answer (label) of a task from unlabeled data and performs supervised training, using the generated label. For example, some words of a sentence are masked, and the neural network is caused to predict the masked words, whereby self-supervised training is performed. A technique of self-supervised training designed for a neural network that predicts molecular energy has also been proposed. In this technique, some atoms constituting a molecule and some bonds between atoms are masked.

Hereinafter, exemplary embodiments according to the disclosed technology will be described with reference to the drawings.

Before describing the details of the embodiments, a problem in self-supervised training designed for a neural network for molecular energy prediction will be described.

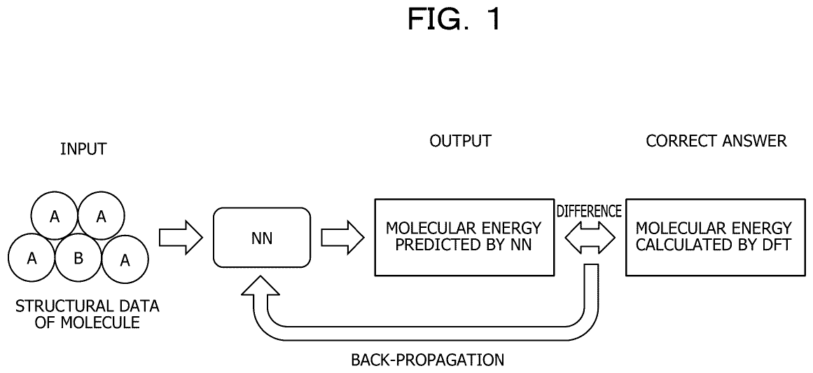

As illustrated in FIG. 1, a neural network (hereinafter, also expressed as “NN”) that predicts energy of a molecule, based on structural data of that molecule is trained by supervised training, using labeled data made up of “structure” and “energy”. For example, the structural data of the molecule is input to the NN, and a parameter of the NN is updated by back-propagating the difference between predicted energy of the molecule, which is the output from the NN, and energy as a correct answer. This energy serving as a correct answer is calculated by, for example, a numerical calculation technique called DFT. Since the DFT is supposed to have a very long calculation time for molecular energy, it is difficult to collect a large amount of labeled data for supervised training.

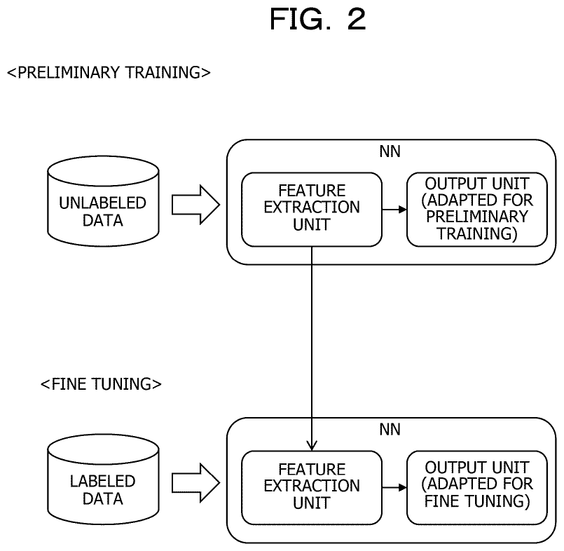

Thus, there is a technique called self-supervised training that generates a correct answer (label) of a task from unlabeled data and performs supervised training, using the generated label. Commonly, self-supervised training is applied to preliminary training in transfer training. As illustrated in FIG. 2, in transfer training, the NN is preliminarily trained by self-supervised training using unlabeled data. The NN includes a feature extraction unit that extracts a feature of data that has been input and an output unit according to a task. An output unit adapted for preliminary training is applied as the output unit at the time of preliminary training.

Then, when the tasks are different between preliminary training and fine tuning, the output unit is altered so as to be adapted for fine tuning. For example, in a case where the task of preliminary training is to classify into three classes and the task of fine tuning is to classify into four classes, the output unit is altered to have a fully connected layer for four-class classification. Thereafter, the NN is trained by supervised training using labeled data adapted for fine tuning. At this time, the feature extraction unit that has been preliminarily trained is applied as it is as the feature extraction unit. By applying self-supervised training as preliminary training, even in a situation in which there is little labeled data to be involved in fine tuning (supervised training) after preliminary training, high accuracy of the NN may be achieved.

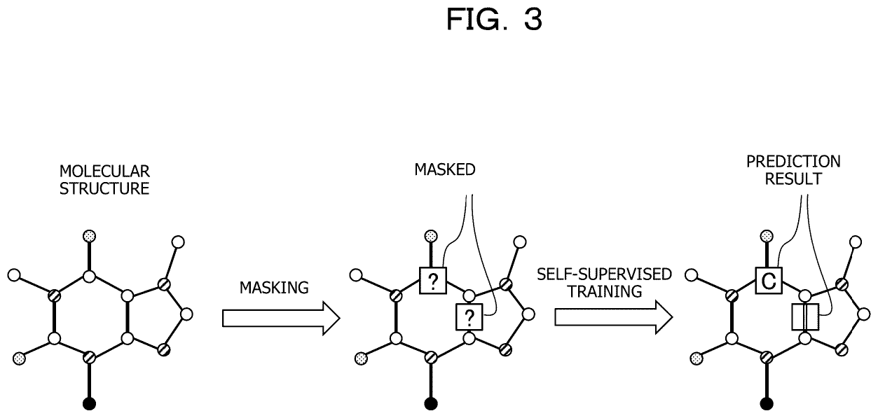

A technique of self-supervised training designed for a neural network that predicts molecular energy has also been proposed. In this technique, as illustrated in FIG. 3, a structure of a molecule represented by atoms constituting the molecule and bonds between atoms is prepared. In the example in FIG. 3, each atom is represented by a circle, and differences in patterns of the circles represent differences in types of the atoms. In the structure of the molecule, a predetermined percentage of atoms or bonded portions is masked, and the NN is caused to predict atoms or bonds at the masked sections. Since the structure of the molecule is known, the correct answers (true atoms or bonds) of the masked sections are already given, and thus self-supervised training is feasible.

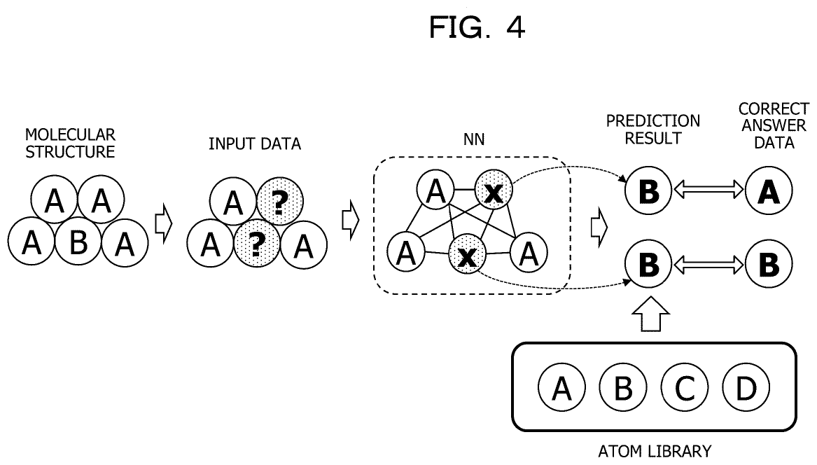

In addition, as illustrated in FIG. 4, the structure of the molecule is represented by input data in which a value (vector) according to an attribute of an atom, such as an element, is allocated to each atom constituting the molecule. In the input data, the atom subjected to masking is replaced with a value according to the masking (expressed as “x” in FIG. 4), such as a value obtained by normalizing a random initial value by the Xavier technique. Then, the NN performs prediction for multi-level classification that predicts which atom in an atom library (in FIGS. 4, A, B, C, and D) prepared beforehand the masked atom is.

However, in the case of self-supervised training in which atoms are masked in this manner, the NN will be trained with a molecular structure including the information on “masking” that does not actually exist, as input data. The value of the masked section in the input data is input to implement masking, and the atom of “masking” does not exist in the atoms constituting the molecule. For this reason, the NN is trained on information on “masking” that is not actually present, and accordingly, useless training is performed, which sometimes deteriorates the prediction accuracy of the trained NN.

Thus, in each of the following embodiments, in a case where self-supervised training of an NN is performed by masking some atoms contained in a molecule, useless training may be restrained from being performed, and prediction accuracy of the NN may be enhanced. Hereinafter, each embodiment will be described in detail.

### First Embodiment

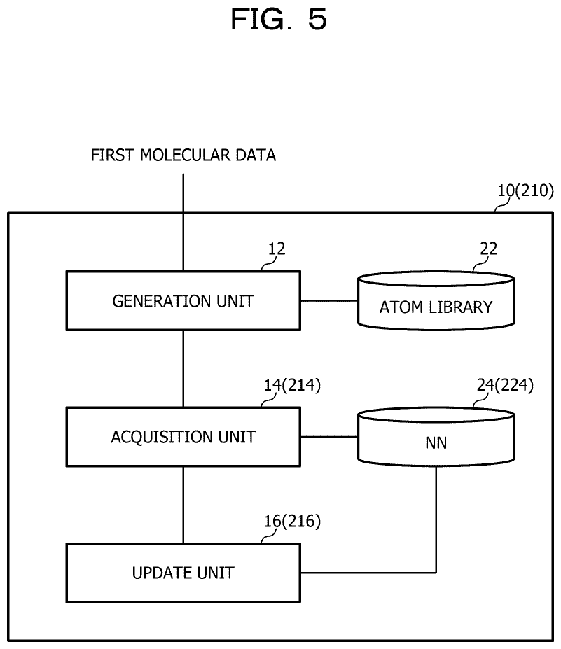

As illustrated in FIG. 5, a self-supervised training device 10 functionally includes a generation unit 12, an acquisition unit 14, and an update unit 16. In addition, an atom library 22 and a neural network (NN) 24 are stored in a predetermined storage area of the self-supervised training device 10. The NN 24 is an example of a “machine learning model” of the disclosed technology. In addition, a case where the NN 24 in the present embodiment is a graph neural network (GNN) will be described.

The generation unit 12 acquires first molecular data indicating a first molecule input to the self-supervised training device 10. The first molecular data is, for example, graph data in which atoms contained in the first molecule are represented by nodes and bonds between atoms are represented by edges coupling between nodes. Each node holds a value (vector) indicating one of atoms corresponding to each node. The generation unit 12 generates second molecular data indicating a second molecule obtained by replacing each of a predetermined percentage of atoms among the atoms contained in the first molecule, with any atom in the atom library 22. The atom library 22 maintains values indicating each atom in a group of a plurality of predefined types of atoms. The atoms whose values are maintained in the atom library 22 are only atoms that actually exist and do not include values corresponding to masking of the previous technologies.

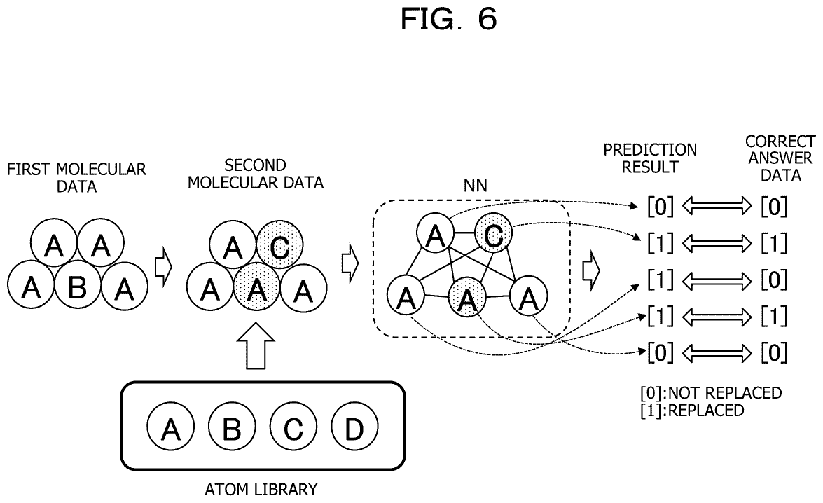

A specific description will be given with reference to FIG. 6. The generation unit 12 specifies, as replacement objects, a number of atoms in accordance with a designated replacement rate from among the atoms contained in the first molecule. The replacement rate has a value from 0 to 100%, where a replacement rate of 0% correlates with a case where there is no replacement, and a replacement rate of 100% correlates with a case where all atoms contained in the molecule are replaced. The example in FIG. 6 depicts a case where the replacement rate is 40%, and two of five atoms contained in the molecule are specified as replacement objects (shaded circles). In addition, the generation unit 12 generates the second molecular data by replacing the value of the node of the atom as a replacement object in the first molecular data with the value of an atom randomly selected from the atom library 22.

The NN 24 includes a feature extraction unit that extracts a feature indicating a molecular structure of the second molecule from the second molecular data, and an output unit that outputs a prediction result according to a task of preliminary training, based on the feature extracted by the feature extraction unit. In the first embodiment, the task of preliminary training is a binary classification task of whether or not each atom contained in the second molecule has been replaced from a relevant one of the atoms contained in the first molecule. In the example in FIG. 6, the prediction result for each atom is represented by [0 (not replaced)] or [1 (replaced)].

The acquisition unit 14 inputs the second molecular data generated by the generation unit 12 to the NN 24 and acquires information indicating whether or not each atom contained in the second molecule has been replaced from the first molecule, which is the prediction result output from the NN 24.

The update unit 16 updates a parameter of the NN 24, based on a comparison result between the correct answer data corresponding to the first molecule and the prediction result acquired by the acquisition unit 14. For example, the update unit 16 generates the correct answer data [0] for atoms corresponding to nodes having the same value between the corresponding nodes in the first molecular data and the second molecular data. In addition, the update unit 16 generates the correct answer data [1] for atoms corresponding to nodes having different values between the corresponding nodes. Then, the update unit 16 back-propagates the difference between the prediction result and the correct answer data to the NN 24 to update a parameter of the NN 24.

The update unit 16 repeats updating of a parameter of the NN 24 until a predetermined end condition is satisfied. For example, the predetermined end condition may be a case where the number of repetitions has reached a predetermined number of times, a case where the difference between the prediction result and the correct answer data has become equal to or less than a predetermined value, a case where the difference between the difference at the last time and the difference at the current time has become equal to or less than a predetermined value (a case where the difference has converged), or the like.

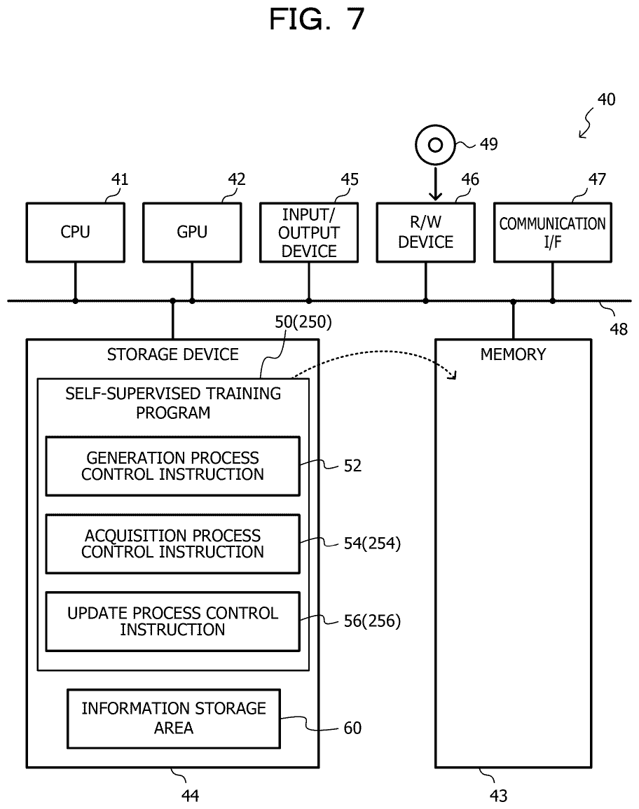

The self-supervised training device 10 may be implemented by, for example, a computer 40 illustrated in FIG. 7. The computer 40 includes a central processing unit (CPU) 41, a graphics processing unit (GPU) 42, a memory 43 as a temporary storage area, and a nonvolatile storage device 44. In addition, the computer 40 includes an input/output device 45 such as an input device and a display device, and a read/write (R/W) device 46 that controls reading and writing of data from and into a storage medium 49. The computer 40 also includes a communication interface (I/F) 47 coupled to a network such as the Internet. The CPU 41, the GPU 42, the memory 43, the storage device 44, the input/output device 45, the R/W device 46, and the communication I/F 47 are coupled to each other via a bus 48.

For example, the storage device 44 is a hard disk drive (HDD), a solid state drive (SSD), a flash memory, or the like. The storage device 44 as a storage medium stores a self-supervised training program 50 for causing the computer 40 to function as the self-supervised training device 10. The self-supervised training program 50 has a generation process control instruction 52, an acquisition process control instruction 54, and an update process control instruction 56. In addition, the storage device 44 includes an information storage area 60 in which information constituting each of the atom library 22 and the NN 24 is stored.

The CPU 41 reads the self-supervised training program 50 from the storage device 44 to load the read self-supervised training program 50 into the memory 43 and sequentially executes the control instructions included in the self-supervised training program 50. The CPU 41 operates as the generation unit 12 illustrated in FIG. 5 by executing the generation process control instruction 52. In addition, the CPU 41 operates as the acquisition unit 14 illustrated in FIG. 5 by executing the acquisition process control instruction 54. In addition, the CPU 41 operates as the update unit 16 illustrated in FIG. 5 by executing the update process control instruction 56. In addition, the CPU 41 reads information from the information storage area 60 and loads each of the atom library 22 and the NN 24 into the memory 43. This ensures that the computer 40 that has executed the self-supervised training program 50 functions as the self-supervised training device 10. Note that the CPU 41 that executes the program is hardware. In addition, a part of the program may be executed by the GPU 42.

Note that the functions implemented by the self-supervised training program 50 may be implemented by, for example, a semiconductor integrated circuit such as an application specific integrated circuit (ASIC) or a field-programmable gate array (FPGA), for example.

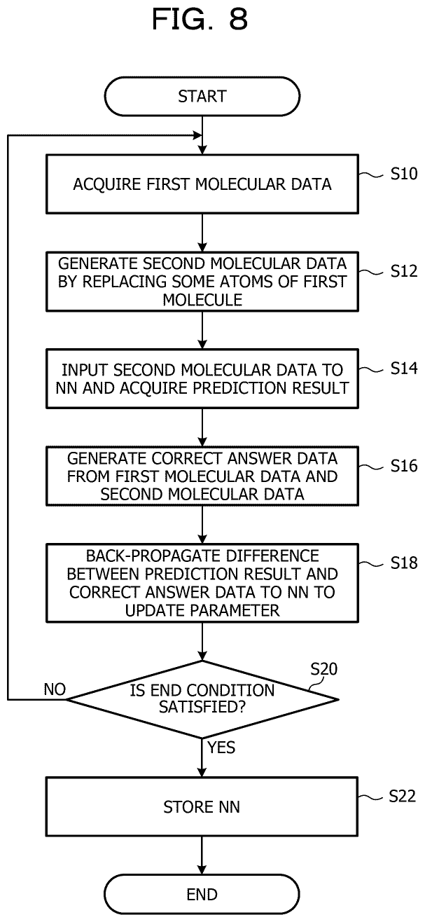

Next, the operation of the self-supervised training device 10 according to the first embodiment will be described. When the first molecular data is input to the self-supervised training device 10 and the execution of the self-supervised training of the NN 24 is prompted, self-supervised training processing illustrated in FIG. 8 is executed in the self-supervised training device 10. Note that the self-supervised training processing is an example of a self-supervised training method of the disclosed technology.

In step S10, the generation unit 12 acquires the first molecular data input to the self-supervised training device 10. Next, in step S12, the generation unit 12 generates the second molecular data indicating the second molecule obtained by replacing each of a predetermined percentage of atoms among the atoms contained in the first molecule, with any atom in the atom library 22. Next, in step S14, the acquisition unit 14 inputs the generated second molecular data to the NN 24 and acquires a prediction result indicating whether or not each of the atoms contained in the second molecule has been replaced from a relevant one of the atoms contained in the first molecule.

Next, in step S16, depending on whether the corresponding nodes have the same value or different values between the first molecular data and the second molecular data, the update unit 16 generates the correct answer data [0 (not replaced)] or [1 (replaced)] for the atoms corresponding to the corresponding nodes. Next, in step S18, the update unit 16 back-propagates the difference between the prediction result and the correct answer data to the NN 24 to update a parameter of the NN 24.

Next, in step S20, the update unit 16 determines whether or not a parameter update end condition is satisfied. In a case where the end condition is satisfied, the processing proceeds to step S22, and in a case where the end condition is not satisfied, the processing returns to step S10. In step S22, the update unit 16 stores the NN 24 set with a final parameter in a predetermined storage area, and the self-supervised training processing ends.

As described above, the self-supervised training device according to the first embodiment generates the second molecular data obtained by replacing each of a predetermined percentage of atoms among the atoms contained in the first molecule, with any atom in the atom library. Then, the self-supervised training device inputs the second molecular data to the NN that performs prediction regarding the molecular structure to acquire a prediction result and updates a parameter of the NN, based on a comparison result between the correct answer data corresponding to the first molecule and the prediction result. In this manner, by replacing the replacement object atom with an atom that actually exists, instead of a random value, useless information training may be restrained, and the prediction accuracy of the neural network trained by self-supervised training may be enhanced.

### Second Embodiment

Next, a second embodiment will be described. Note that, in a self-supervised training device according to the second embodiment, similar components to the components of the self-supervised training device 10 according to the first embodiment will be denoted by the same reference signs, and detailed description will be omitted.

As illustrated in FIG. 5, a self-supervised training device 210 functionally includes a generation unit 12, an acquisition unit 214, and an update unit 216. In addition, an atom library 22 and an NN 224 are stored in a predetermined storage area of the self-supervised training device 210.

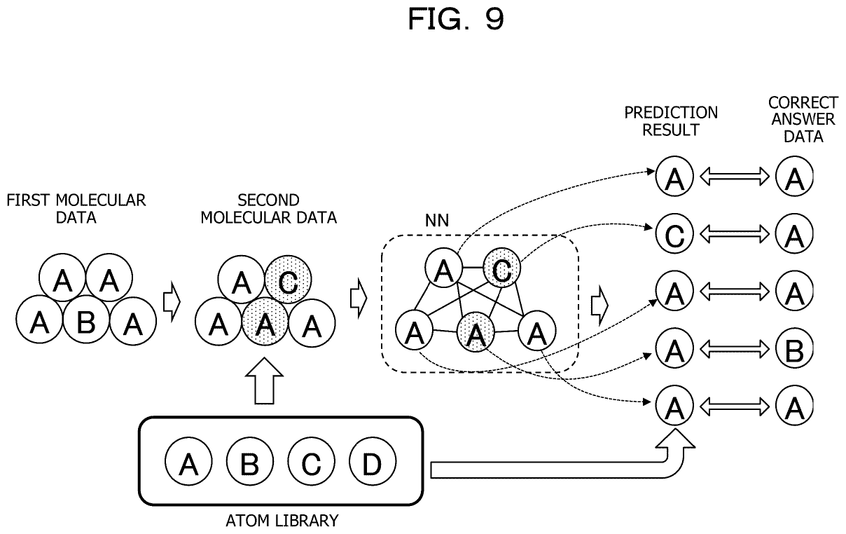

Similarly to the NN 24 of the first embodiment, the NN 224 includes a feature extraction unit that extracts a feature indicating a molecular structure of the second molecule from the second molecular data, and an output unit that outputs a prediction result according to a task of preliminary training, based on the feature extracted by the feature extraction unit. In the second embodiment, as illustrated in FIG. 9, the task of preliminary training is a multi-level classification task of predicting which atom in the atom library 22 each of the atoms contained in the second molecule is.

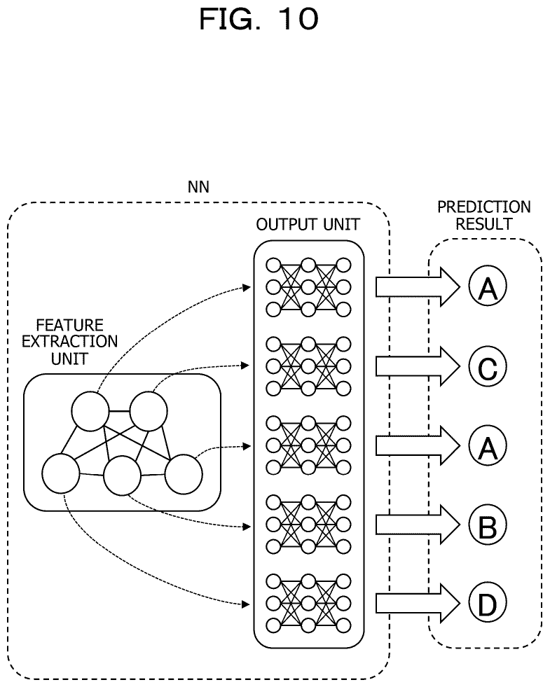

Note that, as illustrated in FIG. 10, in the NN 224, the output unit may be constituted by different fully connected layers, each of which is coupled to output of one of the feature extraction units corresponding to each node constituting the second molecular data that is input data to the NN 224. This allows all the atoms contained in the second molecule to be predicted at one time.

The acquisition unit 214 inputs the second molecular data generated by the generation unit 12 to the NN 224 and acquires information indicating which atom in the atom library 22 each atom contained in the second molecule is, which is the prediction result output from the NN 224.

The update unit 216 updates a parameter of the NN 224, based on a comparison result between the correct answer data corresponding to the first molecule and the prediction result acquired by the acquisition unit 214. For example, the update unit 216 assigns each of the atoms contained in the first molecule as correct answer data and back-propagates a difference between the prediction result and the correct answer data to the NN 224 to update a parameter of the NN 224. The update unit 216 repeats updating of a parameter of the NN 224 until a predetermined end condition is satisfied. The predetermined end condition is similar to the end condition in the case of the first embodiment.

The self-supervised training device 210 may be implemented by, for example, a computer 40 illustrated in FIG. 7. A storage device 44 of the computer 40 stores a self-supervised training program 250 for causing the computer 40 to function as the self-supervised training device 210. The self-supervised training program 250 has a generation process control instruction 52, an acquisition process control instruction 254, and an update process control instruction 256. In addition, the storage device 44 includes an information storage area 60 in which information constituting each of the atom library 22 and the NN 224 is stored.

A CPU 41 reads the self-supervised training program 250 from the storage device 44 to load the read self-supervised training program 250 into a memory 43 and sequentially executes the control instructions included in the self-supervised training program 250. The CPU 41 operates as the generation unit 12 illustrated in FIG. 5 by executing the generation process control instruction 52. In addition, the CPU 41 operates as the acquisition unit 214 illustrated in FIG. 5 by executing the acquisition process control instruction 254. In addition, the CPU 41 operates as the update unit 216 illustrated in FIG. 5 by executing the update process control instruction 256. In addition, the CPU 41 reads information from the information storage area 60 and loads each of the atom library 22 and the NN 224 into the memory 43. This ensures that the computer 40 that has executed the self-supervised training program 250 functions as the self-supervised training device 210.

Note that the functions implemented by the self-supervised training program 250 may be implemented by, for example, a semiconductor integrated circuit such as an ASIC, an FPGA, for example.

Next, the operation of the self-supervised training device 210 according to the second embodiment will be described. When the first molecular data is input to the self-supervised training device 210 and the execution of the self-supervised training of the NN 224 is prompted, self-supervised training processing illustrated in FIG. 8 is executed in the self-supervised training device 210. Hereinafter, differences in the self-supervised training processing according to the second embodiment from the self-supervised training processing according to the first embodiment will be described.

In step S14, the acquisition unit 214 inputs the second molecular data to the NN 224 and acquires information indicating which atom in the atom library each atom contained in the second molecule is, which is the prediction result output from the NN 224. Next, in step S16, the update unit 216 assigns each of the atoms contained in the first molecule as correct answer data.

As described above, the self-supervised training device according to the second embodiment acquires information indicating which atom in the atom library each atom contained in the second molecule is, as the prediction result of the NN. As in the first embodiment, this may restrain useless information training and enhance the prediction accuracy of the neural network trained by self-supervised training.

In addition, the NN trained by the self-supervised training device according to each of the above embodiments can be applied to transfer training as described with reference to FIG. 2. For example, the feature extraction unit of the NN trained by the self-supervised training is used as it is, and the output unit is altered in line with the task of molecular energy prediction. Then, for example, an NN for the task of energy prediction is constructed by training the above NN by supervised training with labeled data in which the energy calculated by the DFT is assigned as the correct answer.

Here, validation results for effects of each of the above embodiments will be described. In this validation, each of the following techniques were validated. Comparative Technique 1: a technique of supervised training of an NN only with labeled data (without self-supervised training); Comparative Technique 2: a technique of supervised training of an NN trained by existing self-supervised training (masking with a random value) with labeled data; Present Technique 1: the technique according to the first embodiment; Present Technique 2: the technique according to the second embodiment

In this validation, 3770 pieces of labeled data, which is a combination of the structure of the molecule and the energy corresponding to that structure, were prepared, and among the 3770 pieces of data, 2870 pieces were assigned as training data, and the remaining 900 pieces were assigned as validation data not used for training. In addition, data of all the structures of the 2870 pieces of training data was used for self-supervised training, and data and energy of all the structures of the 2870 pieces of training data were used for supervised training. Furthermore, 5-fold cross validation was used as a validation method.

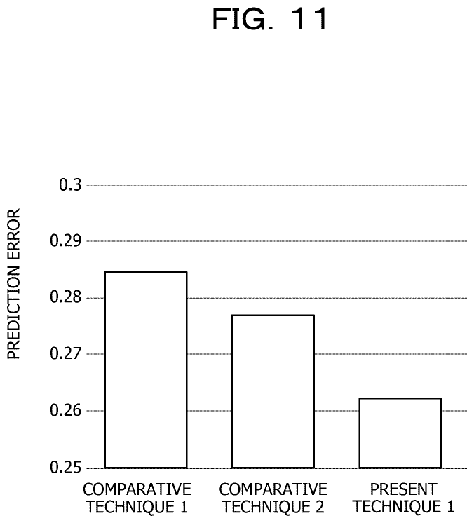

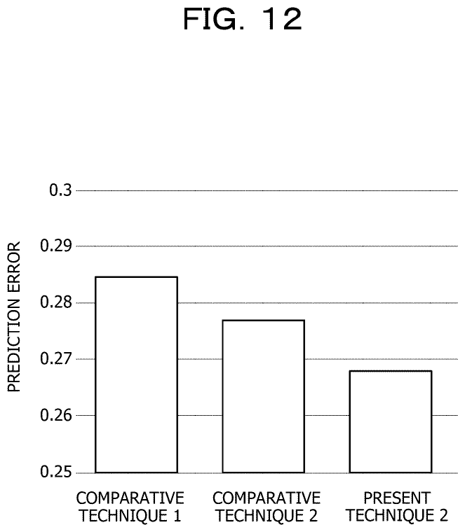

FIG. 11 illustrates a result of comparing prediction errors of the NN between the present technique 1 and the comparative techniques 1 and 2, and FIG. 12 illustrates a result of comparing prediction errors of the NN between the present technique 2 and the comparative techniques 1 and 2. A smaller value of the prediction error represents a better accuracy of the NN. In both of the cases of the present techniques 1 and 2, it can be seen that the prediction errors are reduced and the accuracy is enhanced as compared with the comparative techniques 1 and 2.

In addition, whether or not the accuracy of the NN is improved as compared with the comparative techniques, by applying the self-supervised training of the present technique, using 1660 pieces of the labeled data, even when the number of pieces of the labeled data used at the time of fine tuning is decreased was validated. In this validation, the prediction error of the NN was measured when the number of pieces of the labeled data used at the time of fine tuning was changed. As the labeled data mentioned above, 1660 pieces of data of combinations of the structure of the molecule and the energy corresponding to that structure were prepared, and among the 1660 pieces of data, 1360 pieces were assigned as training data, and the remaining 300 pieces were assigned as validation data not used for training. In addition, data (unlabeled) of all the structures of the 1360 pieces of the training data was used for self-supervised training.

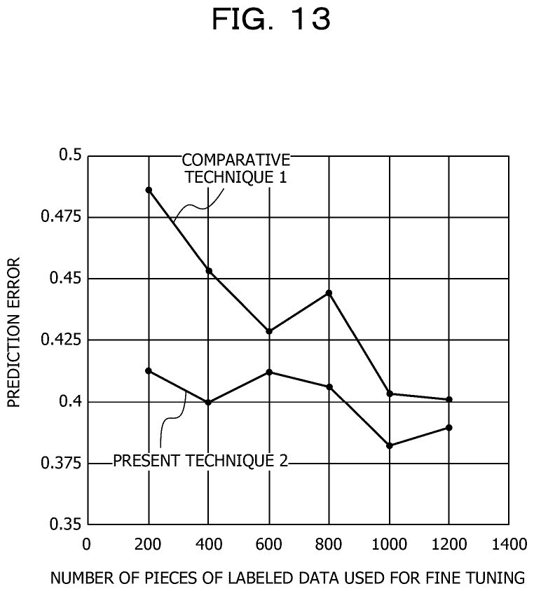

FIG. 13 illustrates a result of comparing prediction errors with respect to the number of pieces of the labeled data used for fine tuning between the present technique 2 and the comparative technique 1. It can be seen that the prediction errors are reduced in the present technique 2 as compared with the comparative technique 1. For example, in a situation with a smaller number of pieces of the labeled data, the present technique 2 has a larger improvement effect on the accuracy of the NN.

Note that, in each of the above embodiments, a case of acquiring the prediction result for all the atoms contained in the second molecule has been described. However, the prediction result may be acquired by assuming at least an atom of the second molecule replaced from an atom contained in the first molecule, as a prediction object. However, since the task becomes more difficult as the number of prediction objects increases, the prediction accuracy of the trained neural network is enhanced.

In addition, while the self-supervised training program is stored (installed) beforehand in the storage device in each of the embodiments described above, the embodiments are not limited to this. The program according to the disclosed technology may be provided in a form stored in a storage medium such as a compact disc read only memory (CD-ROM), a digital versatile disc read only memory (DVD-ROM), or a universal serial bus (USB) memory.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A non-transitory computer-readable recording medium storing a self-supervised training program for causing a computer to execute a process comprising:
generating data that indicates a second molecule obtained by replacing each of a predetermined percentage of atoms among the atoms contained in a first molecule, with any of the atoms included in a group of a plurality of predefined types of the atoms;
acquiring a prediction result by inputting the data that indicates the second molecule to a machine learning model that performs prediction regarding a molecular structure; and
updating a parameter of the machine learning model, based on a comparison result between correct answer data that corresponds to the first molecule and the prediction result.

2. The non-transitory computer-readable recording medium according to claim 1, wherein
the prediction result is information that indicates whether or not at least the atoms replaced from the atoms contained in the first molecule, among the atoms contained in the second molecule, are replaced from each of the atoms contained in the first molecule, and
the correct answer data is data that indicates a difference between the data that indicates the first molecule and the data that indicates the second molecule.

3. The non-transitory computer-readable recording medium according to claim 1, wherein
the prediction result is information that indicates which of the atoms contained in the group of the atoms at least the atoms replaced from the atoms contained in the first molecule, among the atoms contained in the second molecule, are, and
the correct answer data is the data that indicates the first molecule.

4. The non-transitory computer-readable recording medium according to claim 2, wherein the prediction result includes the information on all the atoms contained in the second molecule.

5. The non-transitory computer-readable recording medium according to claim 1, wherein
when the predetermined percentage is zero, the data that indicates the second molecule is the data that indicates the first molecule, and
the prediction result includes information on all the atoms contained in the second molecule.

6. The non-transitory computer-readable recording medium according to claim 1, wherein the machine learning model includes: a first portion that extracts a feature that indicates the molecular structure of the second molecule, from the data that indicates the second molecule; and a second portion that outputs the prediction result according to a specified task, based on the feature extracted by the first portion, and the first portion of the trained machine learning model is used for transfer training.

7. The non-transitory computer-readable recording medium according to claim 6, wherein
the second portion includes different output portions that each correspond to an output of the first portion for each of the atoms contained in the second molecule, and
the acquiring the prediction result includes acquiring the prediction result for each of the atoms contained in the second molecule at one time.

8. A self-supervised training method comprising:
generating data that indicates a second molecule obtained by replacing each of a predetermined percentage of atoms among the atoms contained in a first molecule, with any of the atoms included in a group of a plurality of predefined types of the atoms;
acquiring a prediction result by inputting the data that indicates the second molecule to a machine learning model that performs prediction regarding a molecular structure; and
updating a parameter of the machine learning model, based on a comparison result between correct answer data that corresponds to the first molecule and the prediction result.

9. The self-supervised training method according to claim 8, wherein
the prediction result is information that indicates whether or not at least the atoms replaced from the atoms contained in the first molecule, among the atoms contained in the second molecule, are replaced from each of the atoms contained in the first molecule, and
the correct answer data is data that indicates a difference between the data that indicates the first molecule and the data that indicates the second molecule.

10. The self-supervised training method according to claim 8, wherein
the prediction result is information that indicates which of the atoms contained in the group of the atoms at least the atoms replaced from the atoms contained in the first molecule, among the atoms contained in the second molecule, are, and
the correct answer data is the data that indicates the first molecule.

11. The self-supervised training method according to claim 9, wherein the prediction result includes the information on all the atoms contained in the second molecule.

12. The self-supervised training method according to claim 8, wherein
when the predetermined percentage is zero, the data that indicates the second molecule is the data that indicates the first molecule, and
the prediction result includes information on all the atoms contained in the second molecule.

13. The self-supervised training method according to claim 8, wherein the machine learning model includes: a first portion that extracts a feature that indicates the molecular structure of the second molecule, from the data that indicates the second molecule; and a second portion that outputs the prediction result according to a specified task, based on the feature extracted by the first portion, and the first portion of the trained machine learning model is used for transfer training.

14. The self-supervised training method according to claim 13, wherein
the second portion includes different output portions that each correspond to an output of the first portion for each of the atoms contained in the second molecule, and
the acquiring the prediction result includes acquiring the prediction result for each of the atoms contained in the second molecule at one time.

15. An information processing device comprising:
a memory and
a processor coupled to the memory and configured to:
generate data that indicates a second molecule obtained by replacing each of a predetermined percentage of atoms among the atoms contained in a first molecule, with any of the atoms included in a group of a plurality of predefined types of the atoms;
acquire a prediction result by inputting the data that indicates the second molecule to a machine learning model that performs prediction regarding a molecular structure; and
update a parameter of the machine learning model, based on a comparison result between correct answer data that corresponds to the first molecule and the prediction result.

16. The information processing device according to claim 15, wherein
the prediction result is information that indicates whether or not at least the atoms replaced from the atoms contained in the first molecule, among the atoms contained in the second molecule, are replaced from each of the atoms contained in the first molecule, and
the correct answer data is data that indicates a difference between the data that indicates the first molecule and the data that indicates the second molecule.

17. The information processing device according to claim 15, wherein
the prediction result is information that indicates which of the atoms contained in the group of the atoms at least the atoms replaced from the atoms contained in the first molecule, among the atoms contained in the second molecule, are, and
the correct answer data is the data that indicates the first molecule.

18. The information processing device according to claim 16, wherein the prediction result includes the information on all the atoms contained in the second molecule.

19. The information processing device according to claim 15, wherein
when the predetermined percentage is zero, the data that indicates the second molecule is the data that indicates the first molecule, and
the prediction result includes information on all the atoms contained in the second molecule.

20. The information processing device according to claim 15, wherein the machine learning model includes: a first portion that extracts a feature that indicates the molecular structure of the second molecule, from the data that indicates the second molecule; and a second portion that outputs the prediction result according to a specified task, based on the feature extracted by the first portion, and the first portion of the trained machine learning model is used for transfer training.
